(19) United States
(12) Patent Application Publication
(10) Pub. No.: US 2026/0027341 A1
(43) Pub. Date: Jan. 29, 2026

(54) APPARATUSES AND METHODS FOR DIRECT CAROTID INTERVENTION

(71) Applicant: THE REGENTS OF THE UNIVERSITY OF CALIFORNIA, Oakland, CA (US)

(72) Inventors: Matthew Amans, San Francisco, CA (US); Kazim Narsinh, San Francisco, CA (US)

(21) Appl. No.: 19/144,077

(22) PCT Filed: Jan. 3, 2024
(86) PCT No.: PCT/US2024/010141

§ 371 Date: Jun. 27, 2025

Related U.S. Application Data
(60) Provisional application No. 63/436,712, filed on Jan. 3, 2023.

(51) Int. Cl. A61M 39/02; A61F 2/01; A61F 2/966; A61M 25/10

(52) U.S. Cl. CPC A61M 39/0247; A61F 2/013; A61F 2/966; A61F 2210/0014; A61M 2025/1079; A61M 2039/0258; A61M 2039/0279; A61M 2039/0297

(57) ABSTRACT

A method of deploying a stent is disclosed. The delivery device includes: an outer sheath including a stent located within, the stent making frictional contact with and exerting outward force on an inner surface of the outer sheath. A first wire located coaxially within the lumen and the stent. The first wire has a distal end extending distally of the outer sheath, and a proximal end. extending proximally of the outer sheath. A filter is disposed within the outer sheath and coupled to the first wire and distally of the stent and a distal tip detachably coupled to the distal end of the outer sheath. The method includes separating the outer sheath from the distal tip and withdrawing to deploy the stent and the filter. The method further includes withdrawing the distal tip to collapse the filter. as well as withdrawing the filter and the distal tip through the stent.

### CROSS-REFERENCE TO RELATED APPLICATION

The present application claims the benefit of and priority to U.S. Provisional Patent Application No. 63/436,712, filed Jan. 3, 2023. The entire disclosure of the foregoing application is incorporated by reference herein.

### BACKGROUND

Standard of care therapy for endovascular interventions including for acute ischemic stroke involves endovascular recanalization typically from a transfemoral approach. Difficulties with navigating the aortic arch sometimes make the transfemoral approach impossible, or inefficient, leading surgeons to the alternate strategy of puncturing the common carotid artery for access to the anterior circulation that bypasses the aortic arch. Currently, the same tools that are used for transfemoral access are also used for direct carotid access in stroke intervention. These tools, which are designed for femoral approach, are not suitable for direct carotid access since they are very cumbersome for surgeons to use, have inappropriate transitions in stiffness for use in direct carotid access, and/or are not sized appropriately. In addition, surgical exposure to the carotid artery for endovascular stenting of carotid atherosclerotic disease may have lower rates of periprocedural stroke by bypassing the aortic arch. There is no device to allow for direct carotid access without surgical exposure. Therefore, there is a need for specialized devices for direct carotid vascular access to perform endovascular interventions including stroke therapy and carotid stenting.

### SUMMARY

According to one embodiment of the present disclosure, a carotid artery access sheath is disclosed. The carotid artery access sheath includes an elongated tubular body having a proximal opening and a tapered distal end portion having a distal opening and a seal disposed over the distal opening. The sheath also includes a first lumen extending through the elongated tubular body from the distal opening to the proximal opening. The sheath further includes a balloon disposed coaxially with and over the elongated tubular body proximally of the tapered distal end portion. The sheath additionally includes a second lumen fluidly isolated from the first lumen and extending through the elongated tubular body and in fluid communication with the balloon, where a fluid is supplied through the second lumen to inflate the balloon.

Implementations of the above embodiment may include one or more of the following features. According to one aspect of the above embodiment, the fluid may be a contrast medium. The carotid artery access sheath may further include a bleed port disposed on the elongated tubular body and a third lumen fluidly isolated from the first lumen and the second lumen. The third lumen extends through the elongated tubular body and in fluid communication with the bleed port.

According to another embodiment of the present disclosure, a stent delivery device is disclosed. The stent delivery device includes an outer sheath having an elongated tubular member having a distal end and a proximal end and defining a lumen. The device also includes a stent located within the outer sheath, the stent making frictional contact with and exerting an outward force on an inner surface of the outer sheath. The device further includes a first wire located coaxially within the lumen and the stent. The first wire includes a distal end extending distally of the distal end of the outer sheath and a proximal end, extending proximally of the proximal end of the outer sheath. The device also includes a filter disposed within the outer sheath and coupled to the first wire and distally of the stent. The outer sheath is configured to be withdrawn proximally to deploy the stent and the filter.

Implementations of the above embodiment may include one or more of the following features. According to one aspect of the above embodiment, the stent delivery device may further include a distal tip detachably coupled to the distal end of the outer sheath and a second wire coupled to the distal tip. The outer sheath may also include a perforation between the distal tip and the outer sheath, which may be configured to be separated from the distal tip at the perforation. The distal tip is movable proximally by pulling on the second wire to withdraw the distal tip. The distal tip may have a conical shape configured to engage and collapse the filter during withdrawal of the distal tip, such that the filter and the distal tip pass through the deployed stent. The stent may be a self-expanding stent formed from a nickel-titanium alloy. The first wire may further include a proximal stop attached thereto, the proximal stop being proximal to a distal end of the stent. The proximal stop may be configured to contact the stent during withdrawal of the outer sheath thereby deploying the stent.

According to a further embodiment of the present disclosure, a method of deploying a stent is disclosed. The method includes inserting a stent delivery device into a blood vessel. The stent delivery device includes: an outer sheath having an elongated tubular member with a distal end and a proximal end and defining a lumen. The device also includes a stent located within the outer sheath, the stent making frictional contact with and exerting an outward force on an inner surface of the outer sheath. The device further includes a first wire located coaxially within the lumen and the stent. The first wire has a distal end extending distally of the distal end of the outer sheath, and a proximal end, extending proximally of the proximal end of the outer sheath.

The device additionally includes a filter disposed within the outer sheath and coupled to the first wire and distally of the stent and a distal tip detachably coupled to the distal end of the outer sheath. The method also includes separating the outer sheath from the distal tip and withdrawing the outer sheath proximally from the blood vessel to deploy the stent and the filter. The method further includes withdrawing the distal tip proximally from the blood vessel to collapse the filter. The method additionally includes withdrawing the filter and the distal tip through the deployed stent.

Implementations of the above embodiment may include one or more of the following features. According to one aspect of the above embodiment, the stent delivery device may further include a second wire coupled to the distal tip. Separating the outer sheath from the distal tip may occur at a perforation between the outer sheath and the distal tip. The distal tip may be withdrawn by pulling on the second wire proximally. The filter and the distal tip may be withdrawn by pulling on the first wire and the second wire, respectively. The distal tip may have a conical shape configured to engage and collapse the filter during withdrawal of the distal tip, such that the filter and the distal tip pass through the deployed stent. The stent may be a self-expanding stent formed from a nickel-titanium alloy. The first wire may further include a proximal stop attached thereto, the proximal stop being proximal to a distal end of the stent and configured to contact the stent during withdrawal of the outer sheath thereby deploying the stent.

According to another embodiment of the present disclosure, stent delivery device is disclosed. The stent delivery device includes an outer sheath having an elongated tubular member with a distal end and a proximal end and defining a lumen. The device also includes a stent located within the outer sheath, the stent making frictional contact with and exerting an outward force on an inner surface of the outer sheath. The device further includes an inner catheter located coaxially within the lumen and the stent. The inner catheter includes a distal end optionally extending distally of the distal end of the outer sheath, and a proximal end, optionally extending proximally of the proximal end of the outer sheath. The device also includes a filter disposed within the outer sheath and coupled to the inner catheter and distally of the stent, where the outer sheath is configured to be withdrawn proximally to deploy the stent and the filter.

Implementations of the above embodiment may include one or more of the following features. According to one aspect of the above embodiment, the stent delivery device may include a distal tip detachably coupled to the distal end of the outer sheath and a plurality of wires interconnecting the outer sheath and the distal tip. The outer sheath may include a perforation between the distal tip and the outer sheath, which is configured to be separated from the distal tip at the perforation. The distal tip may be movable proximally by pulling on the outer sheath to withdraw the distal tip. The distal tip may have a conical shape configured to engage and collapse the filter during withdrawal of the distal tip, such that the filter and the distal tip pass through the deployed stent. The stent may be a self-expanding stent. The self-expanding stent may be formed from a nickel-titanium alloy. The inner catheter may further include a proximal stop attached thereto, the proximal stop being proximal to a distal end of the stent. The proximal stop may be configured to contact the stent during withdrawal of the outer sheath thereby deploying the stent.

According to a further embodiment of the present disclosure, a catheter is disclosed. The catheter includes an outer sheath having an elongated tubular member with a distal end and a proximal end and defining a lumen. The catheter also includes a distal tip coupled to the distal end of the outer sheath insertable through a puncture in a blood vessel. The catheter further includes a plug releasably coupled to the distal tip and positionable by the distal tip within the puncture. The plug includes a plate portion for sealing the puncture and an attachment portion for securing the plug to the blood vessel.

Implementations of the above embodiment may include one or more of the following features. According to one aspect of the above embodiment, the catheter may include an attachment device for securing the plug to the blood vessel. The attachment portion and the attachment device may be configured to threadably couple to each other.

According to an additional embodiment of the present disclosure, a blood leak detection system is disclosed. The blood leak detection system includes a needle sensor device insertable near a sealed vessel puncture. The needle sensor device includes an optical sensor, mechanical, sensor, and/or an electrical sensor for detecting a blood leak. The system also includes an interrogator device coupled to the needle sensor and configured to generate a sensor signal for measuring an electrical property, mechanical property, or an optical property of tissue affected by the blood leak, receive a measurement signal from the needle sensor, compare the measurement signal to a threshold indicative of the blood leak, and output an alert in response to the measurement signal being outside the threshold.

### BRIEF DESCRIPTION OF THE DRAWINGS

Embodiments of the present disclosure are described herein with reference to the accompanying drawings, wherein:

FIG. 1 is a diagram of a patient's brain vasculature;

FIG. 2 a top view of a kit for carotid artery access and treatment according to an embodiment of the present disclosure;

FIG. 3 is side view of an access sheath according to an embodiment of the present disclosure;

FIG. 4 is a transverse, cross-sectional view of the access sheath of FIG. 3 taken along the line 4-4;

FIG. 5 is a cross-sectional view of an access sheath inside a carotid artery according to another embodiment of the present disclosure;

FIG. 6 is a cross-sectional view of the access sheath of FIG. 5;

FIG. 7 is a perspective view of a stent delivery device according to an embodiment of the present disclosure;

FIG. 8 is a perspective, partially uncovered view of a distal end portion of the stent delivery device of FIG. 7;

FIG. 9 is a perspective view of a stent of the stent delivery device of FIG. 7;

FIGS. 10-13 are partial cross-sectional views of the stent delivery device showing the deployment of the stent within the vasculature according to an embodiment of the present disclosure;

FIG. 14 is a perspective, schematic view of another feature the stent delivery device of FIG. 7 according to an embodiment of the present disclosure;

FIG. 15 is a perspective, schematic view of a further feature of the stent delivery device of FIG. 7 according to an embodiment of the present disclosure;

FIG. 16 is a perspective, schematic view of yet another feature of the stent delivery device of FIG. 7 according to an embodiment of the present disclosure;

FIG. 17 is a side view of a plug in a first configuration for sealing a puncture in a blood vessel according to an embodiment of the present disclosure;

FIG. 18 shows the plug in a second configuration and disposed at a distal end portion of the stent delivery device of FIG. 7 according to an embodiment of the present disclosure;

FIG. 19 shows the plug of FIG. 17 is an expanded configuration at the distal end portion of the stent delivery device of FIG. 7 according to an embodiment of the present disclosure;

FIG. 20 shows an attachment device for securing the plug in the expanded configuration to a blood vessel according to an embodiment of the present disclosure;

FIG. 21 shows a tool for securing the attachment device to the plug according to an embodiment of the present disclosure;

FIG. 22 shows a blood leakage detection device according to an embodiment of the present disclosure; and

FIG. 23 shows the blood leakage detection device disposed at the plug implantation site according to an embodiment of the present disclosure.

### DETAILED DESCRIPTION

Embodiments of the present disclosure are described in detail with reference to the drawings, in which like reference numerals designate identical or corresponding elements in each of the several views. As used herein the term “proximal” refers to the portion of a device that is closer to the user, while the term “distal” refers to the portion that is farther from the user. The term “about” denotes a range of ±5% from the stated value.

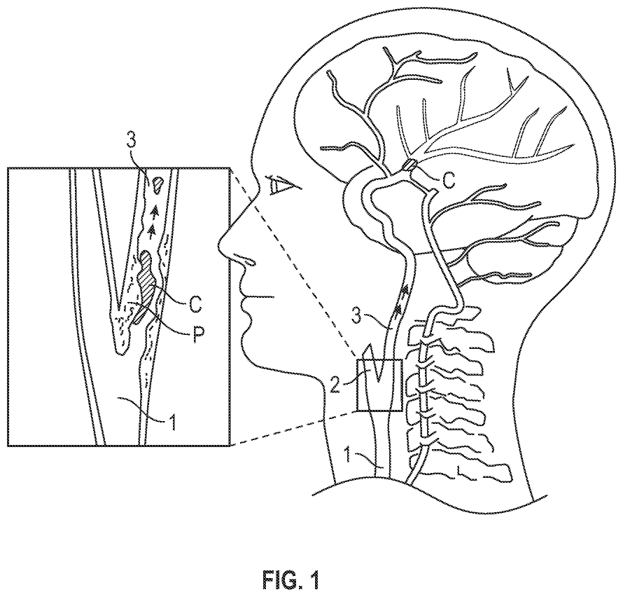

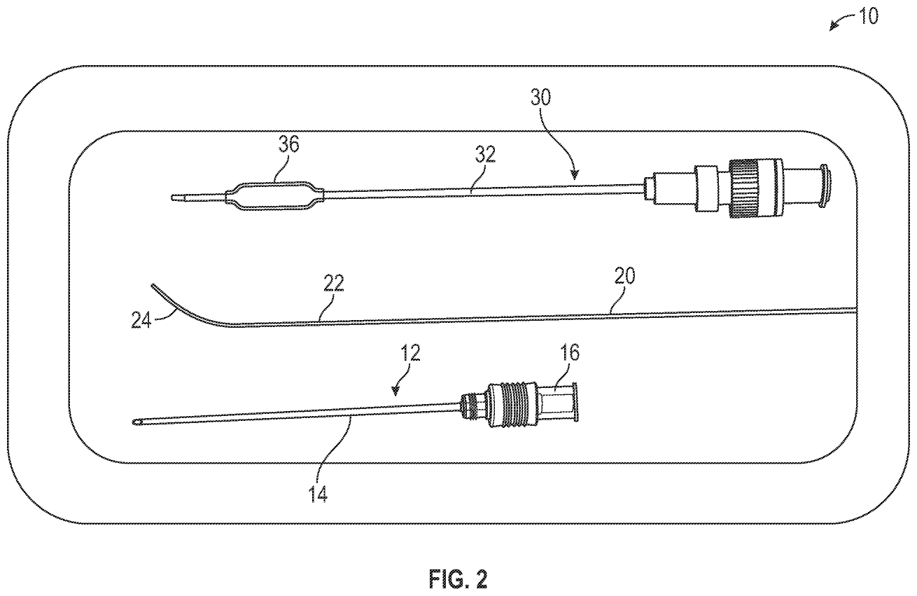

FIG. 1 shows a patient with a common carotid artery 1 splitting into an external carotid artery 2 and an internal carotid artery 3. The patient is also shown as having a clot C in the internal carotid artery 3. The clot C may be disposed at any other blood vessel branching off the common carotid artery 1. The present disclosure provides for a kit 10 shown in FIG. 2 that may be used for over the wire or rapid access of the common carotid artery 1 to treat blockages.

The kit 10 includes an introducer needle 12, which has a needle 14 and a hub 16. The needle 14 may have a gauge from 16 to 21 and may be from about 20 mm to about 60 mm long. The needle 14 may be formed from stainless steel or any other suitable material. The kit 10 also includes a guidewire 20 which may have a diameter from about 0.1 mm to about 0.5 mm and may be from about 10 cm to about 50 cm in length. The introduce needle 12 is used to puncture through the through the patient's neck directly into the common carotid artery 3. The guidewire 20 includes a core portion 22 made from stainless steel or any other suitable metal alloy and includes a flexible distal portion 24, which may be from about 2 cm to about 4 cm. The flexible distal portion 24 may include a coil wound around the core portion 22 and may be formed from nitinol, or any other shape memory metal alloy. The guidewire 20 is inserted through the introducer needle 12 to allow for insertion of additional components of the kit 10 into the common carotid artery.

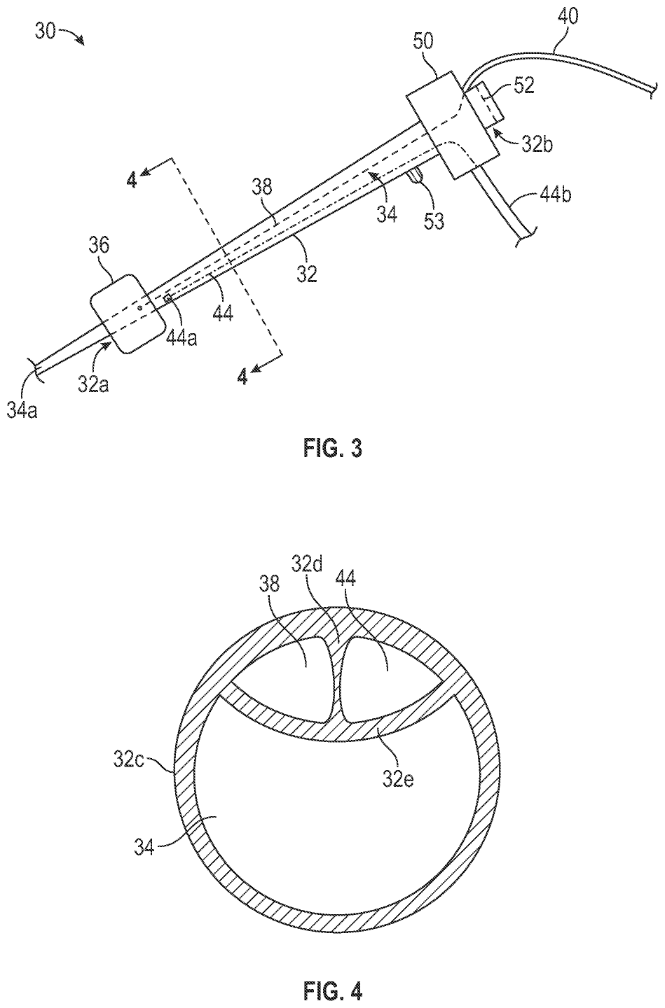

With reference to FIGS. 2 and 3, the kit 10 also includes an access sheath 30 for establishing access through the opening in the common carotid artery. The access sheath 30 includes an elongated tubular body 32 having a distal end portion 32a and a proximal end portion 32b. The tubular body 32 may be formed from any suitable material allowing for insertion of the tubular body 32 into the opening. Suitable materials for the tubular body 32 may have a low coefficient of friction or high lubricity, such as polytetrafluoroethylene, fluorinated ethylene propylene, polyamide, polypropylene, polyethylene, polyethylene terephthalate, polyurethane, polyester, polysiloxanes, etc. Alternatively, any of a variety of lubricious coatings may be applied to the inside and/or outside surface of the tubular body 32, including polytetrafluoroethylene, parylene, and the like. The tubular body 32 may be from about 10 cm to about 50 cm.

The tubular body 32 defines a first lumen 34 extending from the proximal end portion 32b to the distal end portion 32a, which may have a tapered shape to facilitate insertion in the blood vessel. The first lumen 34 may be any suitable diameter to allow for insertion of a stent delivery device 100 therethrough. The first lumen 34 terminates in a distal opening 34a at the distal end portion 32a and a proximal opening 34b. The distal opening 34a may be beveled relative to first lumen 34.

The access sheath 30 also includes an inflatable balloon 36 disposed coaxially with and over the tubular body 32. The balloon 36 is located just proximally of the distal end portion 32a. The balloon 36 may be made of any suitable material, such as, but not limited to, polyethylene terephthalate, copolymers of polyester, polyamide, polyurethane, and copolymers of urethane, and the like. The balloon 36 is in fluid communication with a second lumen 38, which extends from the balloon 36 to the proximal end portion 32b. The balloon 36 is inflated from its first, smaller cross-sectional profile to its second, larger cross-sectional profile following the insertion of the access sheath 30 into a treatment site thereby securing the access sheath 30 in the common carotid artery 1 and preventing blood flow through the opening. The second lumen 38 extends from the balloon 36 to the proximal end portion 32b of the tubular body 32 and is fluidly coupled to an inflation tube 40, which may have a connector (e.g., Luer lock) for coupling to an inflation source, e.g., a syringe filled with contrast fluid.

With reference to FIGS. 3 and 4, the access sheath 30 may further include an optional third lumen 44 having a distal opening 44a disposed proximally of the balloon 36. The lumen 44 extends from the distal opening 44a to the distal end portion 32a of the tubular body 32 to a proximal opening 44b. The distal opening 44a acts as a bleed port to indicate to the operator that the access sheath 30 is positioned within the blood vessel as blood travels through the third lumen 44.

With reference to FIG. 4, the access sheath 30 includes an outer wall 32c and a pair of partition walls 32d and 32e separating the first lumen 34, the second lumen 38, and the third lumen 44. The lumens 34, 38, 44 and the partition walls 32d and 32e may have any cross-sectional shape, e.g., straight, curved, and combinations thereof.

As shown in FIG. 3, the access sheath 30 also includes a hub 50 disposed at a proximal end portion 32b of the tubular body 32. The hub 50 includes a seal 52 blocking the first lumen 34. The hub 50 also includes the proximal opening 34b of the first lumen 34 as well as the inflation tube 40 coupled to the second lumen 38, and a proximal opening 44b of the third lumen 44. The access sheath 30 also includes a fixation attachment 53 disposed between the hub 50 and the balloon 36 allowing for temporarily securing the access sheath 30 to the patient using sutures or another fixation device.

The kit 10 is used for carotid artery interventions and initially includes making a puncture in the common carotid artery 1 or another suitable blood vessel with the introducer needle 12 followed by insertion of the guidewire 20 through the introducer needle 12. The access sheath 30 is then inserted through the opening in the blood vessel and over the guidewire 20. The access sheath 30 is inserted until blood is observed through the third lumen 44. Once it is confirmed that the access sheath 30 is within the common carotid artery, placement of the access sheath 30 may be also confirmed by image fluoroscopy, i.e., position relative to plaque buildup P (FIG. 1). A liquid contrast medium is injected into the second lumen 38 to inflate the balloon 36, the inflated balloon 36 secures the access sheath 30 within the blood vessel. In embodiments, any other fluid may be used to inflate the balloon 36. Contrast media may also be injected through third lumen 44. Once the balloon 36 is inflated and contrast is injected through the distal opening 44a, the area is imaged. If the access sheath 30 is not at a desired position, the balloon 36 is deflated by withdrawing the contrast medium or other fluid, the access sheath 30 is moved and the balloon 36 is reinflated. Imaging may be repeated to confirm placement. Once the access sheath 30 is in place and secured by the balloon 36, the access sheath 30 may be also temporarily secured to the patient with sutures. In addition to providing access to the blood vessel for other instruments, e.g., the stent delivery device 100, the access sheath 30 may be also used to reverse blood flow by applying negative pressure (e.g., using a pump) to the first lumen 34 to prevent dislodged stenosis particles flowing downstream.

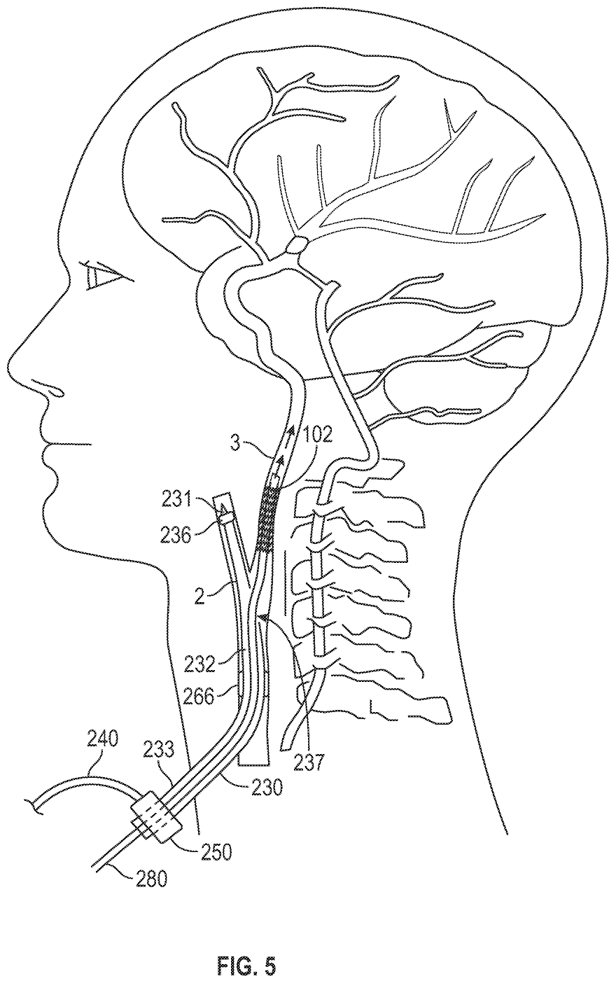

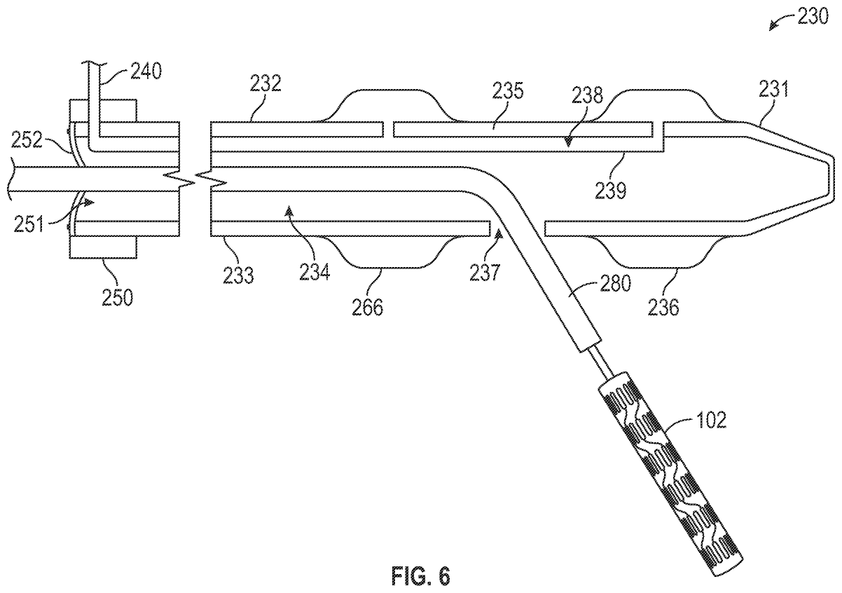

FIGS. 5 and 6 show another embodiment of an access sheath 230 for establishing access through an opening in the common carotid artery 1. The access sheath 230 includes an elongated tubular body 232 having an outer wall 235, a distal end portion 231, and a proximal end portion 233. The distal end portion 231 may have a tapered shape to facilitate insertion in the blood vessel. The tubular body 232 may be formed from any suitable material allowing for insertion of the access sheath 230 into the opening in the common carotid artery 1.

The access sheath 230 includes a distal inflatable balloon 236 and a proximal inflatable balloon 266, which are disposed coaxially with and over the tubular body 232. The balloon 236 is located just proximally, e.g., from about 0.5 cm to about 1 cm, of the distal end portion 231. The balloon 266 is located at approximately the midpoint of the access sheath 230 or from about 8 cm to about 10 cm from the distal balloon 236. The balloons 236 and 266 may be made of any suitable material, such as, but not limited to, polyethylene terephthalate, copolymers of polyester, polyamide, polyurethane, and copolymers of urethane, and the like. The balloons 236 and 266 are in fluid communication with an inflation lumen 238, which extends from the distal balloon 236 to the proximal balloon 266 and to the proximal end portion 233. The inflation lumen 238 is fluidly coupled to an inflation tube 240, which may have a connector (e.g., Luer lock) for coupling to an inflation source, e.g., a syringe filled with contrast fluid.

The tubular body 232 also defines an access lumen 234 extending from the proximal end portion 233 to the distal end portion 231. The access and inflation lumens 234 and 238 may be enclosed by the outer wall 235 and separated by a partition 239 similar to the configuration of the access sheath 30 as shown in FIG. 5. The access lumen 234 may be any suitable diameter to allow for insertion of a stent delivery device 280 through the access lumen 234. The stent delivery device 280 is configured to deliver and deploy the stent 102 and may be any suitable stent delivery device such as carotid WALLSTENT™ from Boston Scientific, of Boston, MA. The access lumen 234 terminates in a side opening 237 through the outer wall 235. The side opening 237 is disposed on the tubular body 232 between the balloons 236 and 266. The side opening 237 may be any suitable size to allow for the stent delivery device 280 to pass therethrough.

The access sheath 230 also includes a hub 250 disposed at a proximal end portion 233 of the tubular body 232. The hub 250 includes a seal 252 blocking the access lumen 234. The hub 250 also includes the proximal opening 251 of the access lumen 234 as well as the inflation tube 240 coupled to the inflation lumen 238.

Following insertion of the access sheath 230 into the common carotid artery 1, the access sheath 230 is advanced until the distal end portion 231 and the distal balloon 236 are within the external carotid artery 2 as shown in FIG. 5. Furthermore, the proximal balloon 266 is disposed within the carotid artery 1 and the side opening 237 is aligned with the interior carotid artery 3 to allow for advancement of the stent delivery device 280 into the internal carotid artery 3. The balloons 236 and 266 are inflated from a first, smaller cross-sectional profile to a second, larger cross-sectional profile. Once balloons 236 and 266 are inflated, the access sheath 230 is secured in the common carotid artery 1 and the external carotid artery 2 and blood flow through the common carotid artery 1 and the external carotid artery 2 is arrested. The stent delivery device 280 is then advanced into the interior carotid artery 3 and the stent 102 is expanded in the interior carotid artery 3.

After deployment of the stent 102, the access sheath 230 may also be used for aspiration. The balloons 236 and 266 arrest all blood flow through the common carotid artery 1 and the exterior carotid artery 2, leaving the interior carotid artery 3 fluidly coupled to the access lumen 234. This configuration allows the access lumen 234 to aspirate the interior carotid artery 3, e.g., to remove any plaque P or clot C materials dislodged by the stent 102. Aspiration may be performed by a negative pressure source coupled to the access lumen 234. The negative pressure source would result in flow reversal and provide for aspiration of the interior carotid artery 3.

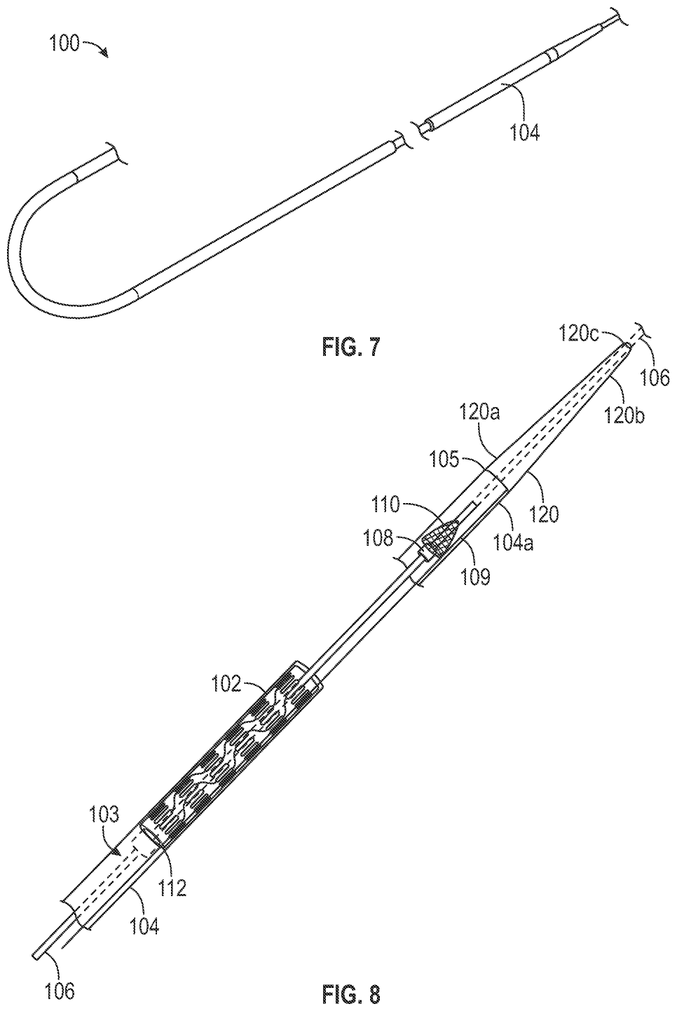

With reference to FIGS. 7 and 8, the kit 10 may also include the stent delivery device 100 configured to deploy a self-expanding stent 102. The stent delivery device 100 includes an outer sheath 104, which is an elongated tubular member having a proximal end portion and a distal end portion 104a and defining a lumen 103. The stent 102 is disposed in an un-expanded, compressed state within the lumen 103 and the outer sheath 104 constrains the stent 102 until the stent 102 is deployed.

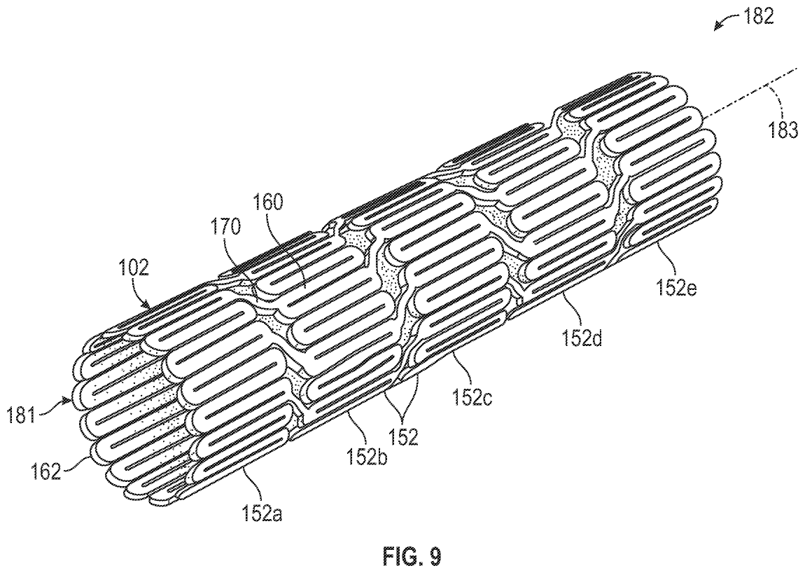

FIG. 9 shows an embodiment of the stent 102 which can be used with the stent delivery device 100. Stent 102 is self-expanding and is shown in its un-expanded compressed state, before being deployed. Stent 102 may be made from an elastic alloy such as nitinol. In embodiments, the stent 102 is made from a memory shape alloy including from about 50.5% by weight of nickel (Ni) to about 60% Ni, and in further embodiments about 55% Ni, with the remainder of the alloy being Ti. The stent 102 is elastic at body temperature, and has an austenite transformation finish temperature (Af) from about 24° C. to about 37° C. The elastic design of the stent 102 makes it crush recoverable allowing it to be used in the carotid artery. As the stent 102 is subjected to temperatures at the austenite phase transformation it attempts to recover to its preformed shape by moving outwardly in a radial direction within the outer sheath 104.

Stent 102 is shaped as a tubular member having proximal and distal open ends 181 and 182, respectively, and a longitudinal axis 183 extending therebetween. In the un-expanded, compressed state as shown in FIG. 9, the stent 102 has a first smaller diameter, suitable for insertion into a patient and navigation through the vessels. In the expanded state as shown in FIGS. 11-13, the stent 102 has a second larger diameter, for deployment into the target area of a vessel. The tubular member is made from a plurality of adjacent hoops 152, FIG. 9 showing hoops 152a-152e, extending between the front and back ends 181 and 182. The hoops 152 include a plurality of longitudinal struts 160 and a plurality of loops 162 connecting adjacent struts, wherein adjacent struts are connected at opposite ends to form an S or Z shape pattern. Stent 102 further includes a plurality of curved bridges 170 which connect adjacent hoops 152. Bridges 170 connect adjacent struts together at bridge-to-loop connection points, which are offset from the center of a loop.

With reference to FIGS. 7 and 8, the stent delivery device 100 also includes a distal tip 120 coupled to a distal end portion 104a of the outer sheath 104. The outer sheath 104 and the distal tip 120 may be formed from any suitable polymeric material, such as polyamide, polyurethane, polytetrafluoroethylene, and polyethylene including multi-layer or single layer structures, and combinations thereof. The distal tip 120 is detachable from the outer sheath 104 at a perforation 105.

The distal tip 120 has a proximal end 120a whose diameter is substantially the same as the outer diameter of the outer sheath 104. The distal tip 120 tapers to a smaller diameter from its proximal end 120a to its distal end 120b, wherein the distal end 120b of the distal tip has a diameter smaller than the inner diameter of the outer sheath 104. The distal tip 120 prevents blood from entering the outer sheath 104 as the device 100 is being navigated through the body vessels. The distal tip 120 also includes a distal opening 120c at the distal end 120a. The distal opening 120a may be beveled relative to the lumen 103.

The distal tip 120 is coupled to one or more pull wires 109 which may be formed from any suitable conformable material, such as stainless steel, and may have a diameter from about 0.05 mm to about 0.2 mm. The pull wires 109 are disposed in the lumen 103 and are passed through the stent 102.

The stent delivery device 100 also includes a central wire 106 disposed inside the lumen 103 and extending through the outer sheath 104, i.e., proximal end of the wire 106 extends proximally of the proximal end of the outer sheath 104 and similarly the distal end of the wire 106 may extend distally of the distal end of the outer sheath 104. The central wire 106 may be made from any suitable conformable material, such as stainless steel, shape memory material, such as nitinol and may have a diameter from about 0.1 mm to about 0.5 mm. The central wire 106 is freely movable through the outer sheath 104 and passes through distal tip 120 and exits through the distal opening 120c. Thus, the distal tip 120 is not coupled to the central wire 106 and is movable along and relative thereto.

The central wire 106 includes a distal stop (e.g., bumper) 108 coupled to a filter 110, which is disposed in an un-expanded, compressed state at the distal end portion 104a of the outer sheath 104, i.e., proximally of the distal tip 120.

The distal stop 108 is proximal to the distal tip 120 and is distal of the stent 102. The filter 110 may be disposed at a distance from about 2 cm to about 4 cm from the distal end 182 of the stent 102. The distal stop 108 can be made from any suitable material, including stainless steel, and/or from a highly radiopaque material such as platinum, gold tantalum, radiopaque filled polymer, and the like. The distal stop 108 is attached to the central wire 106 by mechanical means, adhesive bonding, or any other suitable method.

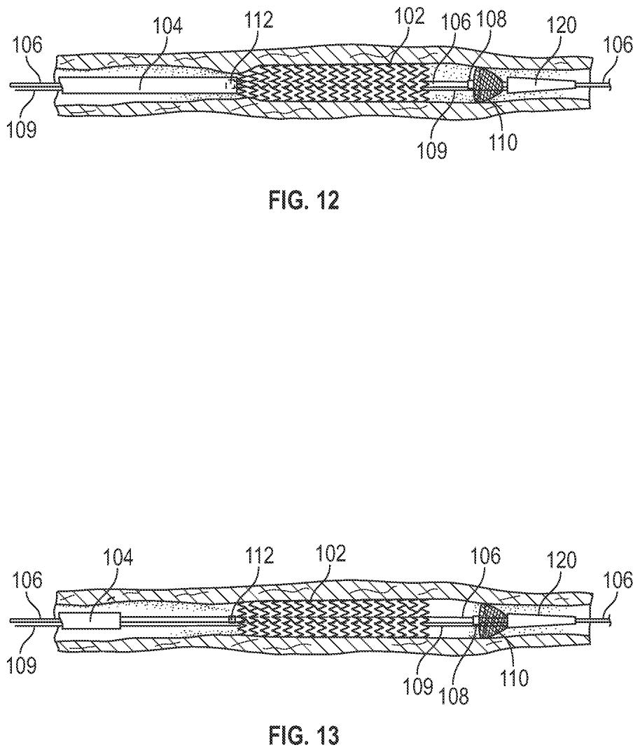

The filter 110 is formed as a porous wire mesh and in the un-expanded, compressed state as shown in FIG. 8, the filter 110 has a first smaller diameter, suitable for insertion into a patient and navigation through the vessels. In the compressed state, the stent 102 is frictionally engaged with an inner surface of the outer sheath 104. In the expanded state as shown in FIGS. 11 and 12, the filter 110 has a second larger diameter, for deployment into the target area of a vessel. The filter 110 may have a conical or tubular shape and may have a plurality of interconnected struts supporting the wire mesh.

The wire 106 also includes a proximal stop (e.g., bumper) 112 disposed proximally of the stent 102 and configured to maintain the stent 102 in place while the outer sheath 104 is pulled back. In particular, the diameter of stop 112 is large enough to make sufficient contact with the loaded stent 102 at its proximal end 181 (FIG. 8) without fully engaging the inner layer of the outer sheath 104. The proximal stop 112 helps to push the stent 102 out of the outer sheath 104 during deployment, by preventing the stent 102 from migrating proximally within the outer sheath 104 during retraction thereof for stent deployment. During deployment, the access sheath 30 is moved in a proximal direction relative to the wire 106 holding the stent 102 in place. The proximal stop 112 may also include radiopaque material, which aids in positioning of the stent 102 within the vessel.

In embodiments, the central wire 106 may be decoupled from the filter 110 and the central wire 106 may be inserted into the outer sheath 104 to push the filter 110 out with the distal stop 108 and the stent 102 via the proximal stop 112.

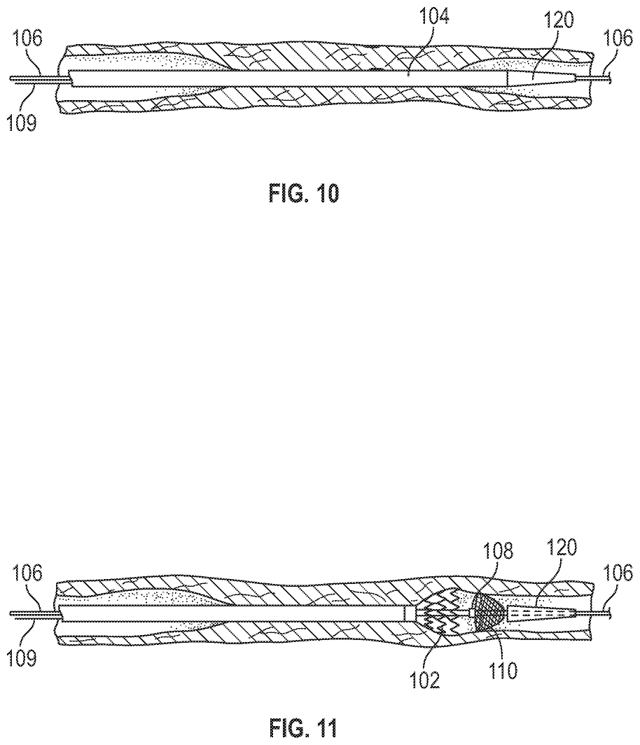

With reference to FIGS. 10-13, the stent delivery device 100 is inserted through the first lumen 34 of the access sheath 30 to access the carotid artery. The stent delivery device 100 is inserted until a desired location is reached and position of the stent 102 relative to the obstruction being treated may be confirmed with image fluoroscopy as shown in FIG. 10. Once location is confirmed, the stent 102 and the filter 110 are deployed by pulling back (i.e., proximally) on the outer sheath 104 to detach the outer sheath 104 from the distal tip 120 along the perforation 105 as shown in FIG. 11. As the outer sheath 104 is removed off the filter 110 and the stent 102, the outer sheath 104 is no longer constraining the filter 110 and the stent 102, allowing them to expand as shown in FIG. 11. The stent 102 in the expanded state pushes against the stenosis and the filter 110 in the expanded state prevents dislodged stenosis particles flowing downstream as shown in FIG. 12. After stent 102 is in place, the filter 110 is collapsed by pulling on the pull wire(s) 109, which draws the distal tip 120 proximally encasing the filter 110 in the conical structure of the distal tip 120 as shown in FIG. 13. The distal tip 120 being pulled proximally reduces the diameter of the filter 110 by collapsing the filter 110 into the cone of the distal tip. After the filter 110 is packed into the distal tip 120, the filter 110 is reduced in size, which allows the filter 110 to pass through the expanded stent 102. The filter 110 is then withdrawn in the collapsed state by pulling on the central wire 106 and the pull wire 109 simultaneously, thereby retracting the distal tip 120 with the filter 110.

In embodiments, the wire 109 may be used to interconnect the distal tip 120 to the outer sheath 104. Rather than being pulled separately from the outer sheath 104, the wire 109 is pulled by the outer sheath 104. Thus, when the outer sheath 104 is withdrawn, the filter 110 and the stent 102 are deployed as described above. As the outer sheath 104 continues to be withdrawn, the distal tip 120 is pulled proximally by the wire 109 attached to the outer sheath 104. The distal tip 120 grabs and retrieves the filter 110 as described above. Thus, the same motion, i.e., pulling on the outer sheath 104, that deploys the stent 102 would be used to deploy and then subsequently retrieve the filter 110 through the expanded stent 102.

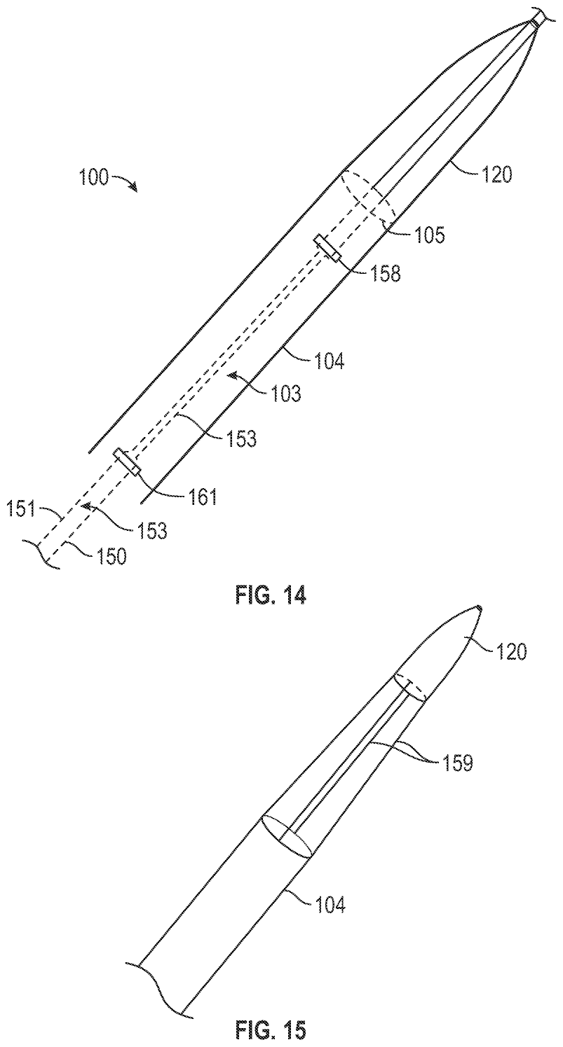

FIG. 14 shows another aspect of the stent delivery device 100, which may include an inner catheter 150 disposed inside the lumen 103 and extending through the outer sheath 104, i.e., proximal end of the inner catheter 150 may extend proximally of the proximal end of the outer sheath 104 and similarly the distal end of the inner catheter 150 may extend distally of the distal end of the outer sheath 104. The inner catheter 150 may replace the central wire 106 and perform the same functions as the central wire 106 as described above, e.g., deploy the stent 102. The inner catheter 150 may have an outer diameter from about 0.1 mm to about 0.5 mm.

The inner catheter 150 is freely movable through the outer sheath 104 and may pass through distal tip 120 and exit through the distal opening 120c. Thus, the distal tip 120 is not coupled to the inner catheter 150 and is movable along and relative thereto. The inner catheter 150 includes a sheath 151 defining a lumen 153. The sheath 151 may be formed any suitable polymeric material, such as polyamide, polyurethane, polytetrafluoroethylene, and polyethylene including multi-layer or single layer structures, and combinations thereof. The guidewire 20 may be inserted into the inner catheter 150 aiding in the insertion of the stent delivery device 100 through the access sheath 30. In embodiments, the stent delivery device 100 may be either an over the wire system as described above with respect to the guidewire 20 or a rapid exchange catheter.

The sheath 151 may include a distal stop (e.g., bumper) 158 coupled to the filter 110, which is disposed in an un-expanded, compressed state at the distal end portion 104a of the outer sheath 104, i.e., proximally of the distal tip 120. The distal stop 158 is proximal to the distal tip 120 and is distal of the stent 102. The filter 110 may be disposed at a distance from about 2 cm to about 4 cm from the distal end 182 of the stent 102. The distal stop 158 can be made from any suitable material, including stainless steel, and/or from a highly radiopaque material such as platinum, gold tantalum, radiopaque filled polymer, and the like. The distal stop 158 is attached to the central inner catheter 150 by mechanical means, adhesive bonding, or any other suitable method. In embodiments, a plurality of filters 110 may be used, which are disposed in series along the central wire 106 or the inner catheter 150. The filters 110 may be deployed and withdrawn by the distal tip 120 in the same manner as described above.

The inner catheter 150 also includes a proximal stop (e.g., bumper) 161 disposed proximally of the stent 102 and configured to maintain the stent 102 in place while the outer sheath 104 is pulled back. In particular, the diameter of stop 112 is large enough to make sufficient contact with the loaded stent 102 at its proximal end 181 (FIG. 8) without fully engaging the inner layer of the outer sheath 104. The proximal stop 161 helps to push the stent 102 out of the outer sheath 104 during deployment, by preventing the stent 102 from migrating proximally within the outer sheath 104 during retraction thereof for stent deployment. During deployment, the access sheath 30 is moved in a proximal direction relative to the inner catheter 150 holding the stent 102 in place. The proximal stop 161 may also include radiopaque material, which aids in positioning of the stent 102 within the vessel.

In embodiments, the inner catheter 150 may be decoupled from the filter 110 and the inner catheter 150 may be inserted into the outer sheath 104 to push the filter 110 out with the distal stop 158 and the stent 102 via the proximal stop 161.

The inner catheter 150 may include a depression 153 (i.e., smaller outer diameter than surrounding areas) for securing the stent 102 to inner catheter 150 prior to deployment of the stent 102. In further embodiments, the inner catheter 150 may also include a balloon (not shown), coupled to an inflation lumen (not shown) to expand the stent 102. The balloon may be disposed on the depression 153 which is used to secure the stent 102. The balloon may be used to expand the stent 102 in embodiments where the stent 102 is not self-expanding.

During use, the stent delivery device 100 may be inserted through the first lumen 34 of the access sheath 30 to access the carotid artery. The guidewire 20 may be used to guide the stent delivery device 100 using the inner catheter 150. The stent delivery device 100 is inserted until a desired location is reached and position of the stent 102 relative to the obstruction being treated may be confirmed with image fluoroscopy as shown in FIG. 10. Once location is confirmed, the stent 102 and the filter 110 are deployed by pulling back (i.e., proximally) on the outer sheath 104 to detach the outer sheath 104 from the distal tip 120 along the perforation 105 as shown in FIGS. 11 and 15. As the outer sheath 104 is removed off the filter 110 and the stent 102, the outer sheath 104 is no longer constraining the filter 110 and the stent 102, allowing them to expand as shown in FIG. 11. The stent 102 in the expanded state pushes against the stenosis and the filter 110 in the expanded state prevents dislodged stenosis particles flowing downstream as shown in FIG. 12.

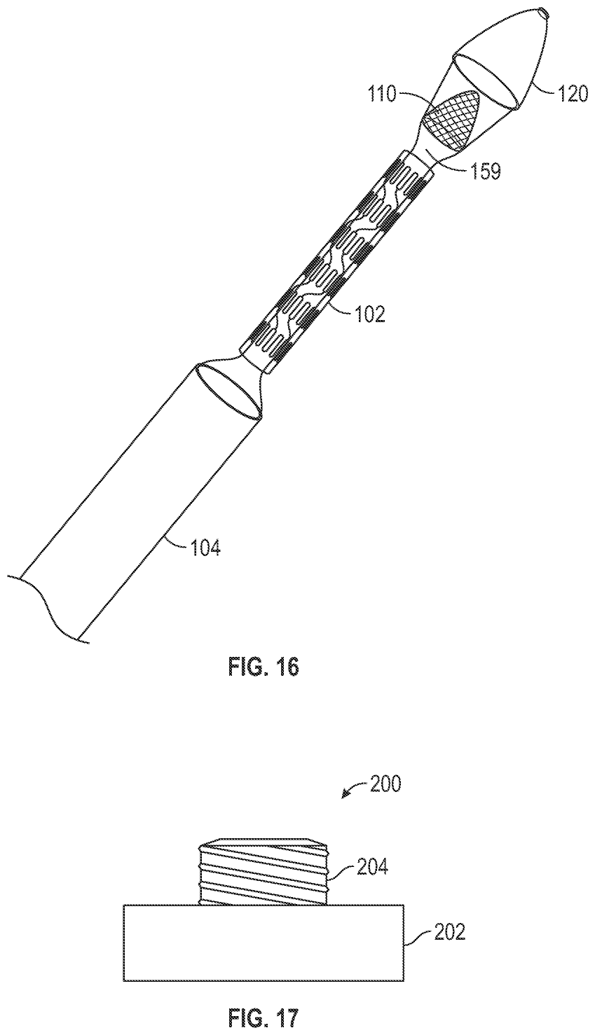

FIG. 15 shows a further aspect of the stent delivery device 100, which may include a plurality of pull wires 159 that interconnect the distal tip 120 to the outer sheath 104. With reference to FIG. 16, the pull wires 159 may be routed through the stent 102, around the filter 110 and attached to the distal tip 120. Rather than being pulled separately from the outer sheath 104, the pull wires 159 are pulled by the outer sheath 104. Thus, after stent 102 is in place, when the outer sheath 104 is withdrawn, the filter 110 and the stent 102 are deployed. As the outer sheath 104 continues to be withdrawn, the distal tip 120 is pulled proximally by the pull wires 159 attached to the outer sheath 104. The distal tip 120 grabs and retrieves the filter 110. The distal tip 120 being pulled proximally reduces the diameter of the filter 110 by collapsing the filter 110 into the cone of the distal tip. The filter 110 draws the distal tip 120 proximally encasing the filter 110 in the conical structure of the distal tip 120 as shown in FIG. 13. Thus, continual motion, i.e., pulling the outer sheath 104 proximally, deploys the stent 102 and then subsequently retrieves the filter 110 through the expanded stent 102. The pull wires 159 may also extend through the length of the outer sheath enabling for retraction of the distal tip 120 as described with respect to the pull wire 109.

After the filter 110 is packed into the distal tip 120, the filter 110 is reduced in size, which allows the filter 110 to pass through the expanded stent 102. The filter 110 is then withdrawn in the collapsed state by continuing to pull on the outer sheath 104, thereby retracting the distal tip 120 with the filter 110. The inner catheter 150 may be withdrawn in tandem with the outer sheath 104.

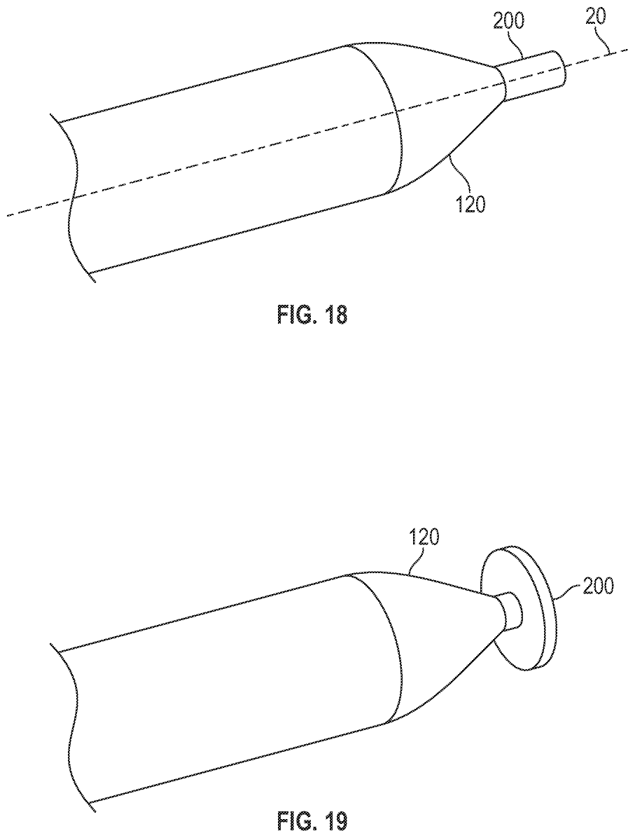

FIG. 17 shows a plug 200 for sealing a puncture formed in a blood vessel, e.g., the carotid artery 3 that was used for introduction of the stent delivery device 100. The plug 200 may be used with the stent delivery device 100 or any other catheter. The plug 200 includes a plate portion 202 and an attachment portion 204 extending from the plate portion 202. In particular, the plate portion 202 is intended to remain inside the blood vessel and the attachment portion 204 is intended to protrude outside the puncture thereby securing the plug 200 to the vessel. The plug 200 may be formed from a porous, bioabsorbable, elastic material, such as oxidized cellulose, hemostatic collagen, polymerized polylactic acid, polyglycolic acid, e.g., poly (glycolide-co-lactide) (PGLA), and combinations thereof. Due to the elastic nature of the material(s) of the plug 200, it may transition between a first (e.g., expanded) configuration as shown in FIGS. 17 and 19-21 and a second (e.g., compressed or bent) configuration as shown in FIG. 18.

As used herein, the terms “biodegradable” and “bioabsorbable” are used with respect to a property of a material. “Biodegradable” is a material that is capable of being decomposed or broken down in vivo and subsequently excreted. “Bioabsorbable” is a material that is capable of being decomposed or broken down in vivo and subsequently resorbed. Both biodegradable and bioabsorbable materials are suitable for purposes of this application and thus for simplicity, unless otherwise directed, biodegradable materials and bioabsorbable materials are collectively referred to as “biodegradable” herein. Conversely, “non-biodegradable” is a biocompatible (i.e., not harmful to living tissue) material that is not decomposed or broken down in vivo. In addition, the term “dissolution” as used in the description refers to the breakdown of both biodegradable and bioabsorbable materials.

With reference to FIGS. 18-21, the plug 200 is secured to a distal end portion of the distal tip 120. In particular, an edge of the plug 200, e.g., of the plate portion 202, may be attached to the distal tip 120 at the edge of the distal opening 120c. The plug 200 is then conformed to the guidewire 20, such as by wrapping the plug 200 around the guidewire 20 as shown in FIG. 18.

In embodiments, the plug 200 may be bent like a flap by the guidewire 20 at attachment point of the plug 200 to the distal tip 120. Prior to bending, the plug 200 may also be folded, rolled, etc. into a smaller shape that is suitable for insertion into the blood vessel by the distal tip 120. Thus, the guidewire 20 is used to maintain the plug 200 in the second configuration.

The plug 200 is deployed after the stent 102 has been expanded and the filter 110 is withdrawn by the distal tip 120. The plug 200 is deployed by positioning the plug 200 within the puncture using the distal tip 120 and then withdrawing the guidewire 20, which allows the plug 200 to transition from the second configuration to the first configuration. As the guidewire 20 is withdrawn, it no longer maintains the plug 200 in the second configuration, e.g., by bending the plug 200 from the distal opening 120c. As a result, the plug 200 bends or unfurls into the first configuration. As the plug 200 is still attached to the distal tip 120, the plug 200 may be positioned to fill the puncture.

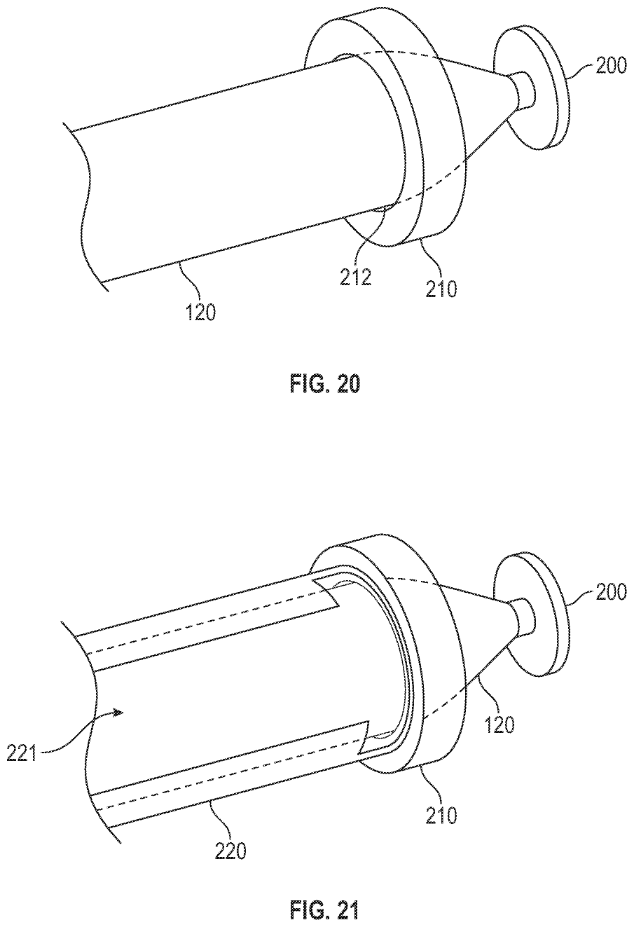

As shown in FIG. 20, the plug 200 may be attached to the surrounding tissue using an attachment device 210 configured to securely couple to the attachment portion 204. The attachment device 210 may include threads, barbs, ridges, and other features configured to securely couple the attachment device 210 to the attachment portion 204. The attachment portion 204 may have a cylindrical shape having a threaded surface as shown in FIG. 17. The attachment device 210 may include an opening 212 having a cylindrical shape configured to receive the attachment portion 204 and may include a counterpart threaded surface. In embodiments, the attachment portion 204 and the opening 212 may have any suitable shape configured to engage each other, e.g., rectangular, hexagonal, etc.

With reference to FIG. 21, the attachment device 210 may be coupled to the plug 200 using an attachment tool 220 (e.g., wrench), which may have inner or outer surface features for rotating the attachment device 210 thereby threadably coupling the attachment device 210 to the plug 200. In embodiments, where other surface features are used, the tool 220 may be used to push the attachment device 210 into engagement with the plug 200. The tool 220 may have a slot 221 for passage of the outer sheath 104 through the tool 220 and allowing for the tool 220 to rotate about outer sheath 104. Once secured, the attachment device 210 and the plug 200 compress the tissue, i.e., wall of the blood vessel. The attachment device 210 may be formed from the same bioabsorbable material(s) as the plug 200. This allows for the plug 200 and the attachment device 210 to be resorbed after a desired period of time, e.g., 2 weeks-2 months.

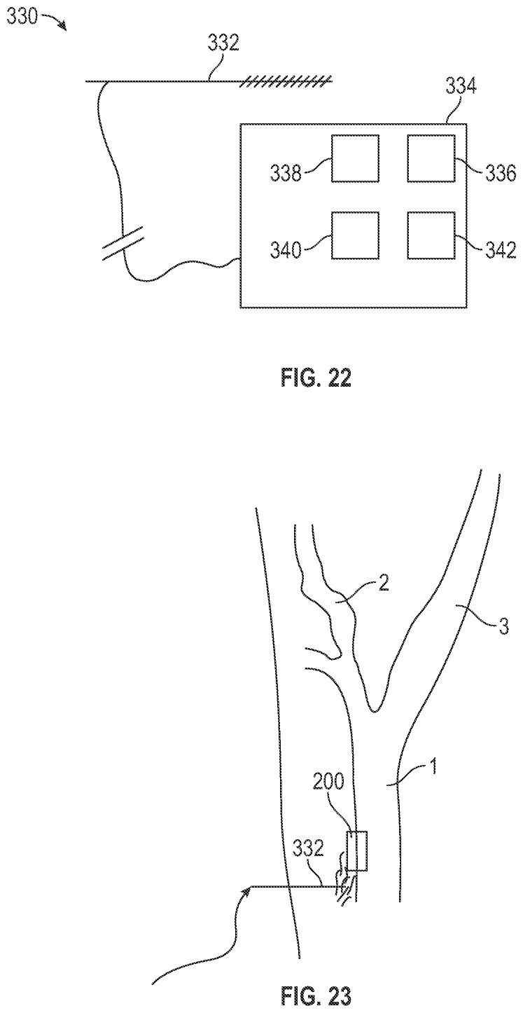

FIGS. 22 and 23 show a leak detector system 330 for detecting whether the hemostasis formed by the plug 200 and the attachment device 210 is leaking. The detector system 330 includes a sensor 332, which may be any blood detecting transducers, such as an electrical, mechanical, or an optical based sensor. An electrical transducer may have one or more electrodes that are configured to output an electrical sensor signal, which may be a radio frequency (RF) alternating current (AC) signal to measure impedance at the sealed puncture site. An optical transducer may be a combination of an optical transmitter and receiver configured to output light at visible and/or near infrared frequencies to detect presence of blood at the sealed puncture site. In particular the optical sensor may be used to measure increase in blood flow. A mechanical transducer may also be used that detects increased pressure changes The sensor 332 may have a needle shape having the electrodes and/or optical elements disposed at the tip of the needle or on a side thereof. The needle shape allows for insertion of the sensor 332 to the sealed puncture site.

The sensor 332 is coupled to an interrogator device 334 via an electrical lead and/or optical fiber. The interrogator device 334 is configured to generate sensor signal, e.g., optical or electrical, measure a change in the sensor signal, and generate an alarm based on the change in the sensor signal indicative of a blood leak. The interrogator device 334 includes a power source 336, which may be a battery, coupled to a signal generator 338, which may be an RF signal generator or a light source (e.g., LED). The signal generator 338 generates a sensor signal, which may be an electrical or optical signal, which is applied by the sensor 332 to the tissue. The measurement signal (i.e., sensor signal affected by the blood leak) from the sensor 332 is received at a signal processor 340 which is configured to compare the signal to a threshold indicative of an increased presence of blood due to a leak. A leak results in additional fluid present at the sensor, which may be detected based on a decrease in impedance. Similarly, increase in presence of blood may be detected based on an increase in a blood parameter (e.g., hemoglobin, oxygenation, etc.) detectable by the optical sensor signal. Thus, a blood leak is detected based on the measurement signal being outside corresponding to normal tissue, i.e., without a blood leak.

The interrogator device 334 also includes an alarm device 342 coupled to the signal processor 340. The alarm device 342 may include one or more audio and/or visual output devices (e.g., speaker, LEDs, screen, etc.), which are configured to output an alert when the blood leak is detected by the signal processor 340 based on the comparison of the measurement signal to a corresponding threshold (e.g., electrical or optical). In embodiments, the interrogator device 334 may be in communication with a patient monitoring device, which may propagate the alert to clinicians, etc.

It will be appreciated that of the above-disclosed and other features and functions, or alternatives thereof, may be desirably combined into many other different systems or applications. Various presently unforeseen or unanticipated alternatives, modifications, variations or improvements may be subsequently made by those skilled in the art which are also intended to be encompassed by the following claims. Unless specifically recited in a claim, steps or components of claims should not be implied or imported from the specification or any other claims as to any particular order, number, position, size, shape, angle, or material.

What is claimed is:

1. A carotid artery access sheath comprising:
an elongated tubular body including a proximal opening and a tapered distal end portion having a distal opening;
a seal disposed over the distal opening;
a first lumen extending through the elongated tubular body from the distal opening to the proximal opening;
a balloon disposed coaxially with and over the elongated tubular body proximally of the tapered distal end portion; and
a second lumen fluidly isolated from the first lumen and extending through the elongated tubular body and in fluid communication with the balloon, wherein a fluid is supplied through the second lumen to inflate the balloon.

2. The carotid artery access sheath according to claim 1, wherein the fluid is a contrast medium.

3. The carotid artery access sheath according to claim 1, further comprising:
a bleed port disposed on the elongated tubular body; and
a third lumen fluidly isolated from the first lumen and the second lumen, the third lumen extending through the elongated tubular body and in fluid communication with the bleed port.

4. A stent delivery device comprising:
an outer sheath including an elongated tubular member having a distal end and a proximal end and defining a lumen;
a stent located within the outer sheath, the stent making frictional contact with and exerting an outward force on an inner surface of the outer sheath;
a first wire located coaxially within the lumen and the stent, the first wire having a distal end extending distally of the distal end of the outer sheath, and a proximal end, extending proximally of the proximal end of the outer sheath; and
a filter disposed within the outer sheath and coupled to the first wire and distally of the stent, wherein the outer sheath is configured to be withdrawn proximally to deploy the stent and the filter.

5. The stent delivery device according to claim 4, further comprising:
a distal tip detachably coupled to the distal end of the outer sheath; and
a second wire coupled to the distal tip.

6. The stent delivery device according to claim 5, wherein the outer sheath includes a perforation between the distal tip and the outer sheath, which is configured to be separated from the distal tip at the perforation.

7. The stent delivery device according to claim 5, wherein the distal tip is movable proximally by pulling on the second wire to withdraw the distal tip.

8. The stent delivery device according to claim 7, wherein the distal tip has a conical shape configured to engage and collapse the filter during withdrawal of the distal tip, such that the filter and the distal tip pass through the deployed stent.

9. The stent delivery device according to claim 4, wherein the stent is a self-expanding stent.

10. The stent delivery device according to claim 9, wherein the self-expanding stent is formed from a nickel-titanium alloy.

11. The stent delivery device according to claim 4, wherein the first wire further includes a proximal stop attached thereto, the proximal stop being proximal to a distal end of the stent.

12. The stent delivery device according to claim 11, wherein the proximal stop is configured to contact the stent during withdrawal of the outer sheath thereby deploying the stent.

13. A method of deploying a stent, the method comprising:
inserting a stent delivery device into a blood vessel, the stent delivery device including:
an outer sheath including an elongated tubular member having a distal end and a proximal end and defining a lumen;
a stent located within the outer sheath, the stent making frictional contact with and exerting an outward force on an inner surface of the outer sheath;
a first wire located coaxially within the lumen and the stent, the first wire having a distal end extending distally of the distal end of the outer sheath, and a proximal end, extending proximally of the proximal end of the outer sheath;
a filter disposed within the outer sheath and coupled to the first wire and distally of the stent; and
a distal tip detachably coupled to the distal end of the outer sheath separating the outer sheath from the distal tip;
withdrawing the outer sheath proximally from the blood vessel to deploy the stent and the filter;
withdrawing the distal tip proximally from the blood vessel to collapse the filter; and
withdrawing the filter and the distal tip through the deployed stent.

14. The method according to claim 13, wherein the stent delivery device further includes a second wire coupled to the distal tip.

15. The method according to claim 14, wherein separating the outer sheath from the distal tip occurs at a perforation between the outer sheath and the distal tip.

16. The method according to claim 14, wherein the distal tip is withdrawn by pulling on the second wire proximally.

17. The method according to claim 14, wherein the filter and the distal tip are withdrawn by pulling on the first wire and the second wire, respectively.

18. The method according to claim 13, wherein the distal tip has a conical shape configured to engage and collapse the filter during withdrawal of the distal tip, such that the filter and the distal tip pass through the deployed stent.

19. The method according to claim 13, wherein the stent is a self-expanding stent formed from a nickel-titanium alloy.

20. The method according to claim 13, wherein the first wire further includes a proximal stop attached thereto, the proximal stop being proximal to a distal end of the stent and configured to contact the stent during withdrawal of the outer sheath thereby deploying the stent.

21. A stent delivery device comprising:
an outer sheath including an elongated tubular member having a distal end and a proximal end and defining a lumen;
a stent located within the outer sheath, the stent making frictional contact with and exerting an outward force on an inner surface of the outer sheath;
an inner catheter located coaxially within the lumen and the stent, the inner catheter having a distal end extending distally of the distal end of the outer sheath, and a proximal end; and
a filter disposed within the outer sheath and coupled to the inner catheter and distally of the stent, wherein the outer sheath is configured to be withdrawn proximally to deploy the stent and the filter.

22. The stent delivery device according to claim 21, further comprising:
a distal tip detachably coupled to the distal end of the outer sheath; and
a plurality of wires interconnecting the outer sheath and the distal tip.

23. The stent delivery device according to claim 22, wherein the outer sheath includes a perforation between the distal tip and the outer sheath, which is configured to be separated from the distal tip at the perforation.

24. The stent delivery device according to claim 22, wherein the distal tip is movable proximally by pulling on the outer sheath to withdraw the distal tip.

25. The stent delivery device according to claim 24, wherein the distal tip has a conical shape configured to engage and collapse the filter during withdrawal of the distal tip, such that the filter and the distal tip pass through the deployed stent.

26. The stent delivery device according to claim 21, wherein the stent is a self-expanding stent.

27. The stent delivery device according to claim 26, wherein the self-expanding stent is formed from a nickel-titanium alloy.

28. The stent delivery device according to claim 21, wherein the inner catheter further includes a proximal stop attached thereto, the proximal stop being proximal to a distal end of the stent.

29. The stent delivery device according to claim 28, wherein the proximal stop is configured to contact the stent during withdrawal of the outer sheath thereby deploying the stent.

30. A catheter comprising:
an outer sheath including an elongated tubular member having a distal end and a proximal end and defining a lumen;
a distal tip coupled to the distal end of the outer sheath insertable through a puncture in a blood vessel; and
a plug releasably coupled to the distal tip and positionable by the distal tip within the puncture, the plug including a plate portion for sealing the puncture and an attachment portion for securing the plug to the blood vessel.

31. The catheter according to claim 30, further comprising:
an attachment device for securing the plug to the blood vessel.

32. The catheter according to claim 31, further wherein the attachment portion and the attachment device are configured to threadably couple to each other.

33. A blood leak detection system comprising:
a needle sensor device insertable near a sealed vessel puncture, the needle sensor device including at least one of an optical, a mechanical, or an electrical sensor for detecting a blood leak; and
an interrogator device coupled to the needle sensor and configured to:
generate a sensor signal for measuring an electrical, mechanical, or an optical property of tissue affected by the blood leak;
receive a measurement signal from the needle sensor,
compare the measurement signal to a threshold indicative of the blood leak; and
output an alert in response to the measurement signal being outside the threshold.
